(19) United States
(12) Patent Application Publication
(10) Pub. No.: US 2026/0060719 A1
(43) Pub. Date: Mar. 5, 2026

(54) ENTRY SITE TISSUE PUNCTURE SYSTEM

(71) Applicant: ClearPoint Neuro, Inc., Solana Beach, CA (US)

(72) Inventors: Ernesto Salegio, Solana Beach, CA (US); Jeremy Stigall, Solana Beach, CA (US); Princeton Saroha, Solana Beach, CA (US)

(21) Appl. No.: 19/319,044

(22) Filed: Sep. 4, 2025

Related U.S. Application Data
(60) Provisional application No. 63/690,425, filed on Sep. 4, 2024.

(51) Int. Cl. A61B 17/34

(52) U.S. Cl. CPC A61B 17/3478; A61B 17/3421

(57) ABSTRACT

A tissue puncture assembly comprises an elongated needle having a needle distal tip. A cannula has an internal lumen sized and shaped to receive the elongated needle, wherein elongated needle is slidably positionable within the internal lumen via manipulation of a handle. An anchor lumen is coupled to the elongated needle, wherein the anchor lumen is configured to movably receive a tissue anchor.

### CROSS REFERENCE TO RELATED APPLICATIONS

This application claims the benefit of priority under 35 U.S.C. § 119(e) to U.S. Provisional Patent Application Ser. No. 63/690,425, filed Sep. 4, 2024. The disclosure of the application is incorporated by reference in its entirety.

### BACKGROUND

Delivery of biologics to targeted areas of the nervous system requires the localization of a delivery tool, such as a distal tip of the delivery tool, near the biological area of interest or in route to the area of interest. A rigid cannula is an example form factor of such a delivery tool. A lumen of the cannula is used to dose biologics to the area of interest. A common challenge with such procedures is initially using a puncture tool to form a puncture through the tissue leading up to the entry site of interest without requiring an exchange between the puncture tool versus the delivery lumen. Another common challenge is not requiring complete withdrawal of the puncture tool prior to biologics delivery.

A desired state of a puncturing mechanic in conjunction with a biologics delivery system comprises a re-deployable or a reusable puncture tool integrated to the delivery system. Such a system desirably does not require any additional procedural steps such as removing and replacing items through the delivery lumen.

Some potential form factors of the delivery tool include but are not limited to a flexible catheter or a more rigid cannula wherein either device can span various lengths including just past the target tissue or extending further down the vascular system past the targeted tissue. A common challenge with procedures involving such delivery tools is retaining the position of a distal exit point of a delivery lumen (where the dosing occurs) against significant movement within the relative space of the patient's anatomy. Limiting the movement of this lumen of interest and preventing the even worse situation of complete removal of the tool from the target anatomy is an important factor in consistent and accurate targeted dosing, while optimizing the waste of biologics used in a procedure.

### SUMMARY

Disclosed is a tissue puncture tool that has a needle with a distal, multi-plane (such as a tri-plane) needle tip that is manufactured using unique manufacturing fixtures and machining. In an embodiment, at least a distal tip of the needle is manufactured of various materials, including but not limited to stainless steel, titanium, alloys (using the steel and titanium for example), sapphire, or ceramic. The materials can vary based at least on a space or location to which use of the puncture tip is integrated. For example, if used in a Magnetic Resonance (MR) space, an MR compatible material such as titanium/ceramic/sapphire can be used. An MRI safe grade of stainless steels such as 316L stainless steel alloy is also within the scope of this disclosure.

Further disclosed is a delivery system for the tissue puncture tool or a delivery system component that enables safe and secured external anchoring of the device inserted into the anatomy to the patient's epidermis. The delivery system component stops or inhibits unwanted removal of the delivery tool such as during a procedure and prevents significant movement from the external direction that is often associated with patient movement.

In one aspect, there is disclosed a tissue puncture assembly, comprising: an elongated needle having a needle distal tip; a handle on a proximal region of the elongated needle; a cannula having an internal lumen sized and shaped to receive the elongated needle, wherein elongated needle is slidably positionable within the internal lumen via manipulation of the handle; an anchor lumen coupled to the elongated needle, the anchor lumen configured to movably receive a tissue anchor.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows an enlarged view of a cannula and stylet assembly that includes a needle slidably positioned within an internal lumen of an elongated, rigid cannula.

FIG. 2 shows an enlarged view of a needle distal tip protruding distally out of a distal tip of the cannula.

FIG. 3 shows an alternate embodiment of the needle distal tip.

FIG. 4 shows the distal needle tip in a partially retracted state relative to the cannula.

FIG. 5 shows an example cross-sectional view of the distal tip of the needle.

FIG. 6 shows another embodiment of a needle distal tip that forms a sharpened distal edge of the needle.

FIGS. 7 and 8 shows an embodiment wherein the needle distal tip includes a separate tube that forms an infusion lumen.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

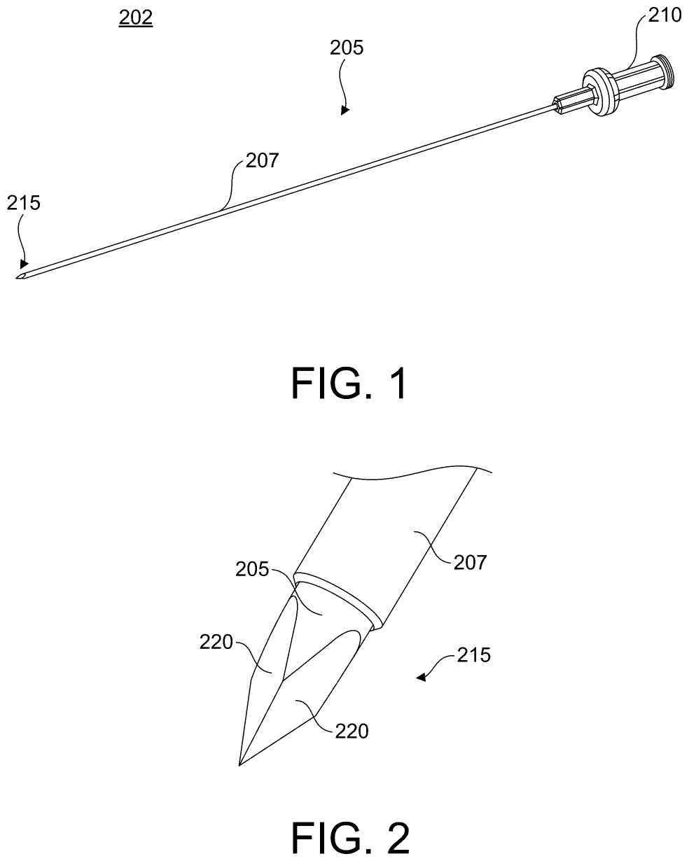

FIG. 1 shows an enlarged view of a guide cannula and stylet assembly that includes a needle 205 (also sometimes referred to as a stylet) slidably positioned within an internal lumen of an elongated, rigid cannula 207. FIG. 1 shows only a distal tip region of the needle 205 which protrudes distally from a distal edge of the cannula 207. The needle is contained co-axially within and covered by the cannula 207. The guide cannula and stylet assembly can be deployed to a biological space (e.g., the cerebrospinal fluid (CSF) space) such as by using a sharpened distal tip 215 of the needle 205 to puncture tissue, such as dura matter of the brain in a non-limiting example. In another non-limiting example, the guide cannula and stylet assembly is positioned in a cannula hub of an external support frame.

With reference still to FIG. 1, the needle 205 is an elongated body that has a proximal handle 210 and a distal needle tip 215. The proximal handle 210 can be used to control co-axial movement of the needle 205 relative to the cannula 207 such that the needle 205 can be extended and retracted relative to the cannula 207. That is, the needle 205 can slide into the cannula through a proximal opening of the cannula such that the distal tip 215 of the needle 205 protrudes distally from a distal edge of the cannula 207. The needle 205 can also be slid entirely or partially out of the cannula 207 such as to vary a position of the distal needle tip 215 relative to the cannula 207. In this manner, the distal tip of the needle 205 can be positioned at various locations relative to a distal tip of the cannula 207. The distal tip 215 of the needle 205 can be moved between an active use, extended state and a safer and inactive, retracted state to prevent unwanted puncture of tissue mid procedure, as well as states therebetween, as described more fully below.

FIG. 2 shows an enlarged view of a distal region of the guide cannula and stylet assembly. The needle distal tip 215 is positioned so that it protrudes distally outward of the distal tip of the cannula 207. The distal tip 215 may be, for example, an inclined, chamfered, pointed or coned region that extends distally from a cylindrical region of the needle. The needle distal tip 215 can be sized and shaped in different manners. The embodiment of the needle distal tip 215 of FIG. 2 has tri-plane configuration, as described more fully below.

A level of retraction or protrusion of the needle's distal tip relative to the cannula distal tip can be varied by sliding the needle 205 relative to the cannula 207. For example, the needle distal tip 215 can be completely “submerged” proximal of and within the distal tip of the cannula 207 such that the inclined region of the distal tip 215 is contained entirely within the cannula. The distal tip 215 can optionally have an atraumatic, tapered edged. Or the needle distal tip 215 can be positioned just below the height level of a deployed anchor (if present) if used in application with a device that employs the use of an internal anchoring system, as described more fully below. The needle distal tip 215 can be aligned optimally past the distal edge of the cannula 207 to form a smooth transition between the two surfaces (e.g., the cannula distal edge and the needle distal tip) when the assembly is in an active puncture state, such as shown in FIG. 2. That is, an outer surface of the needle distal tip is aligned flush with an outer surface of the cannula's distal edge. As described below, the needle 205 can include one or more channels along an entirety or a portion of a length of the needle 205. Such channels can enable movement therethrough of items such as anchoring element(s). The channels can also function as delivery lumen(s).

With reference to FIG. 2, pursuant to the tri-plane configuration, the needle tip has three surfaces 220 that are inclined relative to a long axis of the needle 205. The surfaces 220 extend distally from a cylindrical portion of the needle 205. The three surfaces 220 collectively form an outer surface around a circumference or outer perimeter of the needle's distal region. The surfaces circumferentially converge to form a pointed or rounded distal-most tip of the needle 205. In the illustrated embodiment, the three surfaces 220 form respective planes inclined at 15 degrees relative to a long axis of the needle 205 to form the pointed needle tip 215. It should be appreciated that the needle tip 215 can vary and can include, for example, additional surfaces at various planes and in various angles to disperse a puncture force onto different angular contact surface areas (such as 5-40 degrees).

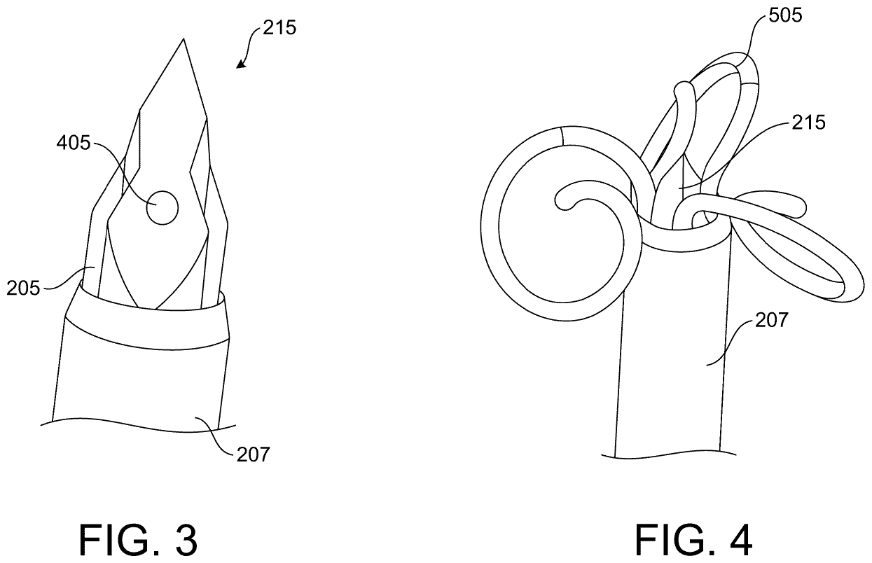

FIG. 3 shows an alternate embodiment of the needle distal tip 215 wherein the needle distal tip 215 includes an aperture 405 or tunnel that extends from one side of the distal tip to another side of the distal tip. The aperture 405 can communicate with an internal lumen of the needle 205 to form a port such as for deploying a tissue anchor or anchor element, which can vary in structure. The internal lumen of the needle can extend along an entire length or a portion of the length of the needle. FIG. 4 shows the needle distal tip 215 in a partially retracted state relative to the distal edge of the cannula 207. That is, a proximal portion of the needle distal tip 215 is contained at least partially within the cannula 207 with a distal portion of the needle distal tip protruding distally out of the cannula 207. An anchor element 505 is deployed out of the distal tip of the needle 205 or out of the distal region of the cannula 207. This embodiment of the anchor element 505 is a wire having an elongated proximal region sized and shaped to fit through a respective lumen. The anchor element has a distal region that forms a fixed, uniform or random series of curves configured to engage and anchor with tissue when deployed. The anchor element 505 can be for example, one or more undulating rods that expand outward from the distal end of the cannula 207. The undulating rods form one or more loops, curly Q, “curlicue”, pig tail, and/or spiral shapes configured to interact and anchor with anatomical tissue.

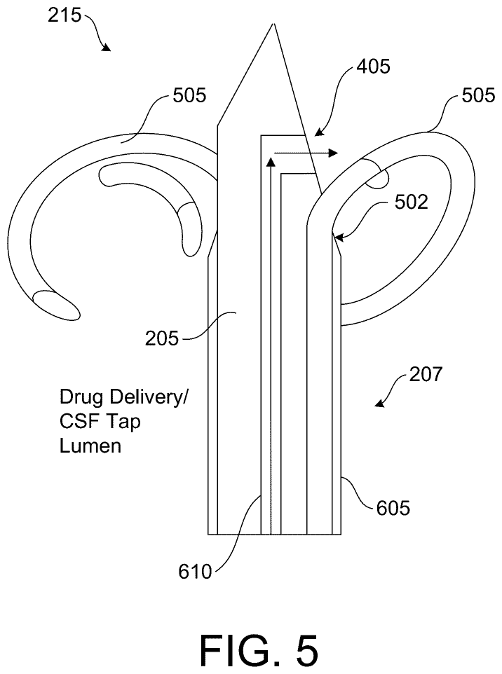

FIG. 5 shows an example cross-sectional view of the needle distal tip 215 positioned with the cannula 207. The needle 205 includes or is coupled to one or more internal channels, lumens, slots or passageways through which aspiration and/or injection can occur and/or through which an elongated device can pass such as the anchor. At least one channel 605 extends lengthwise along a side region of the needle 205 and forms a distal opening 502. In an example implementation, the channel 605 is offset from a central long axis of the needle such that the channel 605 is a side channel. The channel 605 movably houses the anchor element 505 such that the anchor element 505 slides along/within the channel 605 with a distal region of the anchor element protruding out of the cannula. Another channel 610 extends through the needle 205 and serves as an aspiration or injection channel via the aperture 405 (also shown in FIG. 4). In an implementation, the channel 610 is co-axially aligned with a central long axis of the needle (or it can be a second side channel.) The channels can be formed in any of a variety of manners. In an embodiment, at least one channel is formed by a hypotube that extends along a length of the needle such as in a side-by-side relationship to the needle or internal to the needle 205. A delivery lumen (for infusion, aspiration, and/or anchor delivery can also be a space between the cannula and the elongated needle. The lumen or channel can provide for orientations of the anchor or other device that completely parallel to the entry site puncture or 90 degrees relative to the entry site puncture.

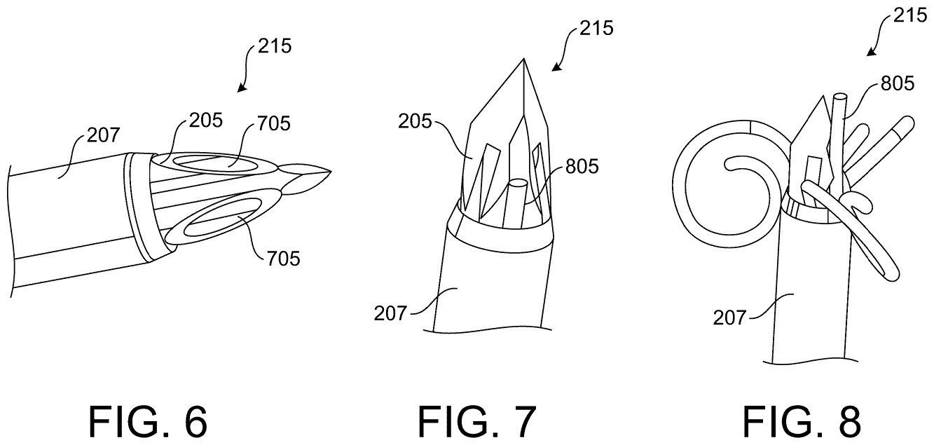

FIG. 6 shows another embodiment of a needle distal tip 215 that forms a sharpened distal edge of the needle 205. The needle distal tip 215 also includes one or more front-facing (or distal facing) delivery openings 705 for injection/infusion and/or for deploying an anchor element and/or therapy. The needle distal tip 215 has one more inclined surfaces that are inclined relative to a long axis of the needle. The opening(s) 705 are positioned on the inclined surfaces.

FIGS. 7 and 8 shows an embodiment wherein the needle tip is made of a primary material. The system includes at least one or more separate polymeric or fused silica tube(s) 805 that each forms an infusion lumen that can travel independently in a controlled fashion alongside the needle 205 within the inner diameter of the guide cannula 207. The tube 805 may also serve as a lumen for delivery of the anchor. The tube may be positioned in a side-by-side relationship with the elongated needle.

In use, an incision is formed in the patient's skin. A guide catheter and/or the guide cannula is passed through the incision so that the distal tip of the cannula and/or guide catheter is a desired location, such as just adjacent to the dura of the brain for example. The needle is then positioned through the cannula so that the distal tip of the needle punctures through the dura. The needle can then be pulled out of the cannula and the cannula is then advanced through the dura to a target location. The anchor is then deployed to the target location. In an embodiment, the anchor is deployed through the needle or side-by-side with the needle while the needle is still positioned within the cannula. In an implementation, the tube can slide relative to the elongated needle. In another implementation, the tube is fixed relative to the elongated needle.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A tissue puncture assembly, comprising:
an elongated needle having a needle distal tip;
a handle on a proximal region of the elongated needle;
a cannula having an internal lumen sized and shaped to receive the elongated needle, wherein elongated needle is slidably positionable within the internal lumen via manipulation of the handle;
an anchor lumen coupled to the elongated needle, the anchor lumen configured to movably receive a tissue anchor.

2. The assembly of claim 1, wherein the needle distal tip has a tri-plane configuration.

3. The assembly of claim 2, wherein the tri-plane configuration includes three surfaces that are inclined relative to a long axis of the needle to form a pointed distal most tip of the needle.

4. The assembly of claim 3, wherein the three surfaces form respective planes inclined at 15 degrees relative to a long axis of the needle to form a pointed needle tip.

5. The assembly of claim 1, wherein the needle distal tip has a plurality of surfaces at various planes and in various angles to disperse a puncture force of the needle distal tip onto different angular contact surface areas.

6. The assembly of claim 1, wherein the anchor lumen is formed by a channel between the elongated needle and the cannula when the elongated needle is positioned within the internal lumen of the cannula.

7. The assembly of claim 6, wherein the channel.

8. The assembly of claim 1, wherein the anchor lumen is formed by a hypotube.

9. The assembly of claim 1, wherein the needle is at least partially manufactured of stainless steel, titanium, sapphire, ceramic, or an alloy.

10. The assembly of claim 1, further comprising the tissue anchor.

11. The assembly of claim 1, wherein the tissue anchor is a wire having an elongated proximal region sized and shaped to fit through the anchor lumen.

12. The assembly of claim 1, wherein the tissue anchor has a distal region that forms a fixed, uniform or random series of curves configured to engage and anchor with tissue when deployed.

13. The assembly of claim 1, wherein the tissue anchor comprises one or more undulating rods that expand outward.

14. The assembly of claim 13, wherein the undulating rods form one or more loops or spiral shapes configured to interact and anchor with anatomical tissue.

15. The assembly of claim 1, wherein the anchor lumen is a tube positioned in a side-by-side relationship with the elongated needle.

16. The assembly of claim 15, wherein the tube can slide relative to the elongated needle.

17. The assembly of claim 1, wherein the anchor lumen is configured for infusion or aspiration of therapy.

18. The assembly of claim 17, wherein the anchor lumen is configured for receiving an elongated anchor.

19. The assembly of claim 1, wherein the anchor lumen is a channel positioned inside the elongated needle.

20. The assembly of claim 1, wherein the anchor lumen is positioned outside the elongated needle.

21. The assembly of claim 20, wherein the anchor lumen is a space between the cannula and the elongated needle.
